(19) United States
(12) Patent Application Publication
(10) Pub. No.: US 2026/0013783 A1
(43) Pub. Date: Jan. 15, 2026

(54) EPILEPTIC SEIZURE DETECTION USING DYNAMIC NETWORK BRAIN MODEL ENTROPY

(71) Applicant: THE JOHNS HOPKINS UNIVERSITY, Baltimore, MD (US)

(72) Inventors: Amir Hossein DARAIE, Baltimore, MD (US); Luis A. SANCHEZ, Baltimore, MD (US); Sridevi V. SARMA, McClean, VA (US); Joon Yi KANG, Baltimore, MD (US)

(21) Appl. No.: 19/269,225

(22) Filed: Jul. 15, 2025

Related U.S. Application Data
(60) Provisional application No. 63/671,315, filed on Jul. 15, 2024.

(51) Int. Cl. A61B 5/00; A61B 5/374

(52) U.S. Cl. CPC A61B 5/4094; A61B 5/374; A61B 5/4836; A61B 5/7264

(57) ABSTRACT

Techniques for automatically detecting a seizure of a patient are presented. The techniques include: obtaining patient EEG data, where the patient EEG data represents an EEG of the patient for a plurality of channels, each channel representing a respective location in or on a brain of the patient; evaluating a tendency to act as a sink for each channel; determining, for each tendency to act as a sink, a respective energy distribution according to frequency; assessing an entropy of each energy distribution according to frequency; measuring, from at least one of the plurality of entropy quantifications, at least one sink tendency entropy drop value; identifying, based on the sink tendency entropy drop value, a presence of a patient seizure proximate to a time of a sink tendency entropy drop; and outputting an indication of the patient seizure.

### RELATED APPLICATION

This application claims the benefit of U.S. Provisional Patent Application No. 63/671,315, entitled “EPILEPTIC SEIZURE DETECTION USING DYNAMIC NETWORK BRAIN MODEL ENTROPY,” filed Jul. 15, 2024, which is hereby incorporated by reference in its entirety.

### FIELD

This disclosure relates generally to detecting and characterizing epileptic seizures.

### BACKGROUND

Epilepsy is a debilitating neurological condition that is characterized by recurrent seizures, profoundly impacting the quality of lives of an estimated 50 million people world-wide. While antiseizure medications can control seizures in many cases, approximately 30% of patients suffer from drug-resistant epilepsy (DRE) and do not respond to medication. For these patients, surgical intervention to resect the seizure onset zone (SOZ) can be an effective treatment. However, surgery involves precise localization of the SOZ, which requires that clinicians assess EEG signals across the brain over multiple seizure events. Obtaining the appropriate data for clinicians to make accurate SOZ assessment involves in-patient monitoring for up to two weeks, followed by manual monitoring of both scalp electroencephalogram (scalp EEG) and intracranial electroencephalogram (iEEG) data to isolate seizure events from the continuous multi-day recordings. (Scalp EEG acquires data through electrodes on the patient's scalp, whereas iEEG acquires data though electrodes on the patient's brain and/or in the patient's brain. As used herein, “EEG” refers to both scalp EEG and iEEG unless the context requires otherwise.) This SOZ assessment process, representing the current approach, is both time-consuming and costly, relying on skilled technicians and clinicians to detect all seizure events from large amounts of data.

Current tools and techniques employed for detecting and localizing seizures each have inherent limitations. Commercially available tools primarily rely on scalp EEG recordings, which are favored for their non-invasiveness and ease of use. However, these tools do not generalize well to iEEG recordings, limiting their effectiveness in detecting seizures originating from deeper brain regions. A further issue with scalp EEG devices is their lower spatial resolution, which can hinder the accurate detection of complex seizure dynamics.

Pattern recognition methods analyze individual EEG channels for seizure-related patterns, but this approach often misses the complex, distributed nature of seizure activity across multiple brain regions, resulting in incomplete detection. Functional connectivity analysis examines interactions between different brain regions, providing a more holistic view, but faces challenges in robustness and reliability, particularly under varying conditions. Frequency analysis methods, which assess the frequency components of EEG signals, are highly sensitive to noise due to the non-stationary nature of EEG signals and the dynamic, multi-frequency patterns of seizures.

Advanced artificial intelligence and machine learning approaches, such as Support Vector Machines (SVM), decision trees, Convolutional Neural Networks (CNNs), and Long Short-Term Memory (LSTM) networks, lack interpretability, which is important in clinical settings where transparency is required for justifiable medical decisions. Further, these techniques may require a lengthy procedure of obtaining and processing training data from a patient before that may be used to detect a seizure. Emerging methods like Graph Neural Networks (GNNs) and deep learning ensembles show future potential, but these methods require substantial computational resources and further validation. Despite these advancements, real-time seizure detection systems face challenges related to processing speed and latency, which are critical for timely intervention.

### SUMMARY

According to various embodiments, a computer-implemented method of automatically detecting a seizure of a patient is presented. The method includes: obtaining patient electroencephalogram (EEG) data, wherein the patient EEG data represents an EEG of the patient for a plurality of channels, each channel representing a respective location in or on a brain of the patient; evaluating, for each channel of the plurality of channels, a respective tendency to act as a sink, from which a plurality of tendencies to act as a sink are obtained, wherein each tendency to act as a sink of the plurality of tendencies to act as a sink quantifies a tendency of a respective location in or on the brain of the patient to act as a sink; determining, for each tendency to act as a sink of the plurality of tendencies to act as a sink, a respective energy distribution according to frequency, from which a plurality of energy distributions according to frequency are obtained; assessing an entropy of each energy distribution according to frequency of the plurality of energy distributions according to frequency, from which a plurality of entropy quantifications are obtained; measuring, from at least one of the plurality of entropy quantifications, at least one sink tendency entropy drop value; identifying, based on the at least one sink tendency entropy drop value, a presence of a patient seizure proximate to a time of a sink tendency entropy drop; and outputting an indication of the patient seizure, wherein the indication of the patient seizure comprises an identification of the time.

Various optional features of the above method embodiments include the following. The method may include treating the patient based on the indication of the patient seizure. The method may include identifying, based on the plurality of entropy quantifications, at least one location in or on the brain of the patient that is epileptogenic of the patient seizure; and outputting an identification of the location in or on the brain of the patient that is epileptogenic of the patient seizure. The method may include treating the patient for epilepsy by one of surgical resection, laser ablation, or electrical stimulation, of the location in or on the brain of the patient that is epileptogenic of the patient seizure. The method may include reducing a dimensionality of the at least one sink tendency entropy drop value, from which a reduced dimensionality is obtained; identifying a plurality of clusters of previous seizures of the patient in the reduced dimensionality; classifying the patient seizure as being in one of the clusters of the plurality of clusters, from which a patient seizure classification is obtained; and outputting an indication of the patient seizure classification. Each of the plurality of tendencies to act as a sink may include a respective dynamic network model sink index. The at least one sink tendency entropy drop value may include a time under threshold value. The at least one sink tendency entropy drop value may include an area under threshold value. The at least one sink tendency entropy drop value may include an event activity metric. Each of the plurality of energy distributions according to frequency may include a spectral power density.

According to various embodiments, a system for automatically detecting a seizure of a patient is presented. The system includes: a non-transitory computer readable medium comprising instructions; and at least one electronic processor that executes the instructions to perform operations comprising: obtaining patient electroencephalogram (EEG) data, wherein the patient EEG data represents an EEG of the patient for a plurality of channels, each channel representing a respective location in or on a brain of the patient; evaluating, for each channel of the plurality of channels, a respective tendency to act as a sink, from which a plurality of tendencies to act as a sink are obtained, wherein each tendency to act as a sink of the plurality of tendencies to act as a sink quantifies a tendency of a respective location in or on the brain of the patient to act as a sink; determining, for each tendency to act as a sink of the plurality of tendencies to act as a sink, a respective energy distribution according to frequency, from which a plurality of energy distributions according to frequency are obtained; assessing an entropy of each energy distribution according to frequency of the plurality of energy distributions according to frequency, from which a plurality of entropy quantifications are obtained; measuring, from at least one of the plurality of entropy quantifications, at least one sink tendency entropy drop value; identifying, based on the at least one sink tendency entropy drop value, a presence of a patient seizure proximate to a time of a sink tendency entropy drop; and outputting an indication of the patient seizure, wherein the indication of the patient seizure comprises an identification of the time.

Various optional features of the above system embodiments include the following. The patient may be treated based on the indication of the patient seizure. The operations may further include: identifying, based on the plurality of entropy quantifications, at least one location in or on the brain of the patient that is epileptogenic of the patient seizure; and outputting an identification of the location in or on the brain of the patient that is epileptogenic of the patient seizure. The patient may be treated for epilepsy by one of surgical resection, laser ablation, or electrical stimulation, of the location in or on the brain of the patient that is epileptogenic of the patient seizure. The operations may further include: reducing a dimensionality of the at least one sink tendency entropy drop value, from which a reduced dimensionality is obtained; identifying a plurality of clusters of previous seizures of the patient in the reduced dimensionality; classifying the patient seizure as being in one of the clusters of the plurality of clusters, from which a patient seizure classification is obtained; and outputting an indication of the patient seizure classification. Each of the plurality of tendencies to act as a sink may include a respective dynamic network model sink index. The at least one sink tendency entropy drop value may include a time under threshold value. The at least one sink tendency entropy drop value may include an area under threshold value. The at least one sink tendency entropy drop value may include an event activity metric. Each of the plurality of energy distributions according to frequency may include a spectral power density.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figures, in which:

FIG. 1 illustrates a pipeline for automatically detecting a seizure of a patient, according to the non-limiting example embodiment of a study disclosed herein;

FIG. 2 is a schematic diagram for a method of automatically detecting a seizure of a patient, according to the non-limiting example embodiment of the study disclosed herein;

FIG. 3 depicts a comparative analysis of SISE values in SOZ and Non-SOZ channels during interictal (rest) and seizure phases, according to the non-limiting example embodiment of the study;

FIG. 4 illustrates iEEG signals from multiple channels for two example patients, according to the non-limiting example embodiment of the study;

FIG. 5 shows a distribution of clinically undetected seizures by hour of the day;

FIG. 6 illustrates distinct seizure clusters in low-dimensional space and their corresponding seizure profiles for a particular patient in the study;

FIG. 7 illustrates SOZ localization using an SISE R score according to the non-limiting example embodiment of the study;

FIG. 8 illustrates seizure onset localization performance metrics as evaluated for the study; and

FIG. 9 presents twelve hours of raw EEG data, as well as SISE average graphs for selected detected events, for a particular patient.

### DESCRIPTION OF THE EXAMPLES

Reference will now be made in detail to example implementations, illustrated in the accompanying drawings. Wherever convenient, the same reference numbers will be used throughout the drawings to refer to the same or like parts. In the following description, reference is made to the accompanying drawings that form a part thereof, and in which is shown by way of illustration specific exemplary examples in which the invention may be practiced. These examples are described in sufficient detail to enable those skilled in the art to practice the invention and it is to be understood that other examples may be utilized and that changes may be made without departing from the scope of the invention. The following description is, therefore, merely exemplary.

Some embodiments utilize a patient-specific network model of the brain to quantify network properties and track significant deviations indicative of brain dysfunction. Some embodiment utilize a Dynamic Network Model (DNM), which quantifies important network-level properties of the brain by capturing the interactions and influence dynamics between different brain regions. Some embodiments are able to quantify regional interactions of the brain's network and localize pathological areas within these networks solely from a snapshot of a patient's iEEG recording. Some embodiments analyze a sink index, which can identify network nodes (brain regions) that act as sinks, in that they are heavily influenced by their neighbors and are not greatly influential to their neighbors. Some embodiments analyze a source index, which can identify network nodes (brain regions) that act as sources, in that they heavily influence their neighbors and are not readily influenced by their neighbors.

Some embodiments identify epileptic events by detecting a drastic reduction in the variability of brain regions being sources and sinks among a plurality (e.g., all) iEEG channels. Such a reduction may result from the SOZ channels returning to being strong sinks as the network attempts to stop the seizure by inhibiting the SOZ. Some embodiments utilize a measure of source-sink variability, e.g., a drop in entropy of a tendency to act as a sink, to identify and localize epileptic seizures.

A non-limiting example embodiment is disclosed throughout this disclosure in reference to a study of data from 54 adults with DRE who underwent iEEG monitoring using depth electrodes at six epilepsy centers. The study divided these patients into two cohorts: 11 with long-term continuous iEEG monitoring without surgical intervention and 43 patients with data restricted to seizure snapshots followed by surgical treatments. The study utilized a non-limiting example of an assessment of the entropy of a spectrum of tendencies to act as a sink, referred to herein as Sink Index Spectral Entropy (SISE). For each patient, the study computed the average SISE across all channels in order to detect seizure events. The technique of the study showed a high sensitivity of 0.98 and precision of 0.94, significantly improving seizure detection accuracy compared to existing techniques By analyzing SISE drops across individual channels, the study classified different seizure types and pinpointed specific brain regions involved in the SISE drops, which highly correlated to clinically annotated SOZ regions (85% accurate SOZ identification). While the average SISE across each epileptic network was significantly higher in interictal periods when compared to seizure periods, the SISE during interictal periods was surprisingly nearly constant. The technique of the study addresses the limitations of current techniques and is expected to facilitate more effective surgical treatments. Thus, various embodiments solve several problems present in existing techniques.

A first problem of prior art techniques is that they utilize EEG data, rather than iEEG data, and thus are ineffective in detecting seizures originating from deep brain regions and have spatial resolution, preventing accurate detection of complex seizure dynamics. Some embodiment solve this first problem of the prior art because they utilize iEEG data.

A second problem of prior art techniques is that they often miss the complex, distributed nature of seizure activity across multiple brain regions, resulting in incomplete detection. Some embodiments solve this second prior art problem by evaluating a tendency to act as a sink (e.g., determining a dynamic network model sink index) for each channel of a set of patient EEG data, where each channel is representative of a location in or on the brain. This multi-channel approach accounts for the distributed nature of seizure activity.

A third problem with prior art techniques is that it is limited in robustness and reliability, particularly under varying conditions. Some embodiments solve this third prior art problem by determining, for each tendency to act as a sink, a respective energy distribution according to frequency (e.g., a spectral power density), and by assessing an entropy of each energy distribution according to frequency. The usage of tendencies to act as a sink, energy distribution thereof, and entropy of the energy distribution, allows for robust and reliable predictions.

A fourth problem with prior art techniques is that they are highly sensitive to noise due to the non-stationary nature of EEG signals and the dynamic, multi-frequency patterns of seizures. Some embodiments solve this fourth prior art problem by assessing an entropy of each energy distribution according to frequency, and measuring one or more sink tendency entropy drop values (e.g., a time under threshold value, an area under threshold value, or an event activity metric) for the entropy quantifications. The usage of entropy drop values for energy distributions essentially eliminates the confounding effects of noise.

A fifth problem with prior art techniques is that they lack interpretability, which is important in clinical settings. Some embodiments solve this fifth prior art problem by identifying, based on the sink tendency entropy drop value(s), a presence of a patient seizure proximate to a time of a sink tendency entropy drop. This identification technique is highly interpretable because it associates a sink tendency entropy drop with the brain network attempting to stop a seizure by inhibiting the SOZ.

A sixth problem with prior art techniques is that they require substantial computational resources, and therefore cannot be used for real-time seizure detection, preventing timely intervention. A seventh problem with the prior art is that many AI and machine learning techniques require training data from the patient before being deployed. Some embodiments solve these sixth and seventh prior art problems through the combination of: (1) iEEG data analysis, (2) determining, for each tendency to act as a sink, a respective energy distribution according to frequency (e.g., a spectral power density), (3) assessing an entropy of each energy distribution according to frequency, (4) measuring one or more sink tendency entropy drop values (e.g., a time under threshold value, an area under threshold value, or an event activity metric) for the entropy quantifications, and (5) identifying, based on the sink tendency entropy drop value(s), a presence of a patient seizure proximate to a time of a sink tendency entropy drop.

These and other features and advantages are shown and described presently in reference to the figures.

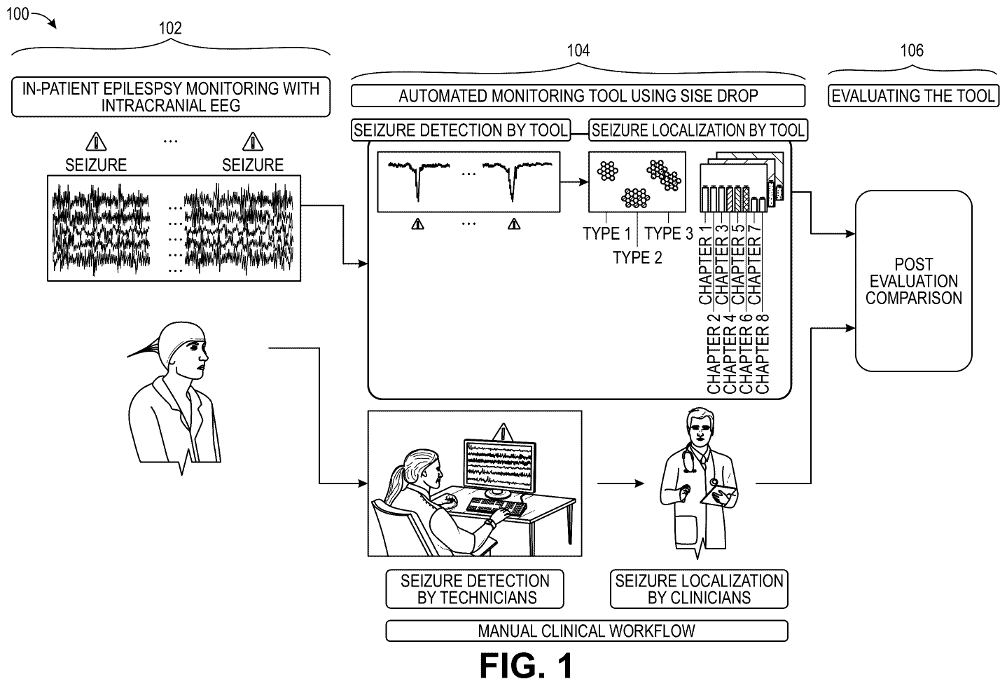

FIG. 1 illustrates a pipeline 100 for automatically detecting a seizure of a patient, according to the non-limiting example embodiment of the study disclosed herein. The study utilized a pipeline to assess the SISE dynamics for seizure detection and localization. The pipeline begins with recording iEEG signals from multiple brain areas during invasive monitoring (102). Clinicians review the iEEG data to identify and categorize seizures (104, bottom row). The embodiment computes the SISE of the iEEG networks, marking significant reductions as seizures. These events are mapped into a lower-dimensional space using Principal Component Analysis (PCA), by way of non-limiting example, where similar seizure events are clustered together. Seizures within the same cluster involve similar brain regions and have the similar duration. Within each seizure cluster, the embodiment determines the involvement of each channel in the seizure by assessing the area under the curve (AUT) and time under the curve (TUT) of the SISE drop (104, top row). Channels highly involved in seizures across all events within the same cluster are more likely to be part of the SOZ. In summary, the embodiment uses SISE scores (a non-limiting example of assessment of the entropy of a tendency to act as a sink) across similar seizures to identify regions likely involved in seizure initiation, providing biomarkers for the SOZ. Finally, a group of board-certified epileptologists evaluated the results for seizure detection and localization (106). When feasible, surgical resection or laser ablation, typically including the SOZ and varying amounts of surrounding tissue, is performed. This process allows for precise seizure detection and localization, aiding in the management and treatment of epilepsy.

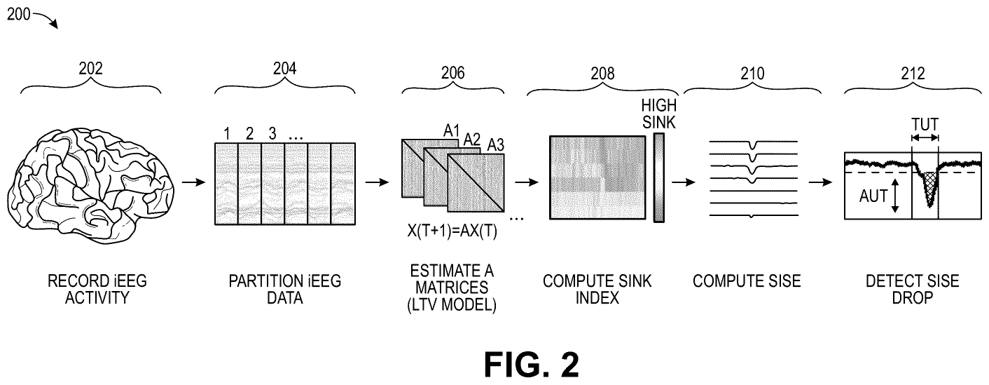

FIG. 2 is a schematic diagram for a method 200 of automatically detecting a seizure of a patient, according to the non-limiting example embodiment of the study disclosed herein. The study involved 54 adults with drug-resistant epilepsy who underwent intracranial EEG (iEEG) monitoring using depth electrodes placed stereotactically. The study collected and analyzed data from two patient groups, both subjected to the same monitoring approach but differing in the extent of recorded data available and subsequent treatments. The two cohorts included one involving long-term continuous iEEG monitoring without surgical intervention and the other focusing on seizure snapshots with subsequent surgical treatments. The first cohort included 11 patients who underwent long-term intracranial EEG (iEEG) monitoring, capturing both interictal (non-seizure or rest) and ictal (seizure) phases, without subsequent surgical intervention. The second cohort included 43 patients whose data were restricted to ictal episodes, including 1 minute before and after seizures. These patients underwent surgical treatments post-monitoring, with options such as resective surgery, laser ablation, or responsive neurostimulation.

At 202, signals were recorded from an N-channel iEEG network and analyzed to construct state transition matrices from 500 ms of data. Stereo-EEG (sEEG) recordings were used in the study. For the collection of sEEG data, Nihon Kohden or Natus (Natus Medical Inc.) EEG monitoring and diagnostic systems were employed, typically operating at a sampling frequency of either 1 or 2 kHz. In a limited number of cases, sEEG data were recorded at a frequency of 500/512 Hz. The positioning of electrodes for each patient was guided by the clinical team at the respective centers. The data underwent bandpass filtering between 0.5 and 120 Hz using a fourth-order Butterworth filter and were further notch filtered at 60 Hz and its harmonics with a 2 Hz stopband. To determine the final electrode locations, information was merged from co-registered postimplantation CT and brain MRI scans, utilizing tools like Biolmage Suite. The clinical teams across each center conducted a visual confirmation of the electrode localizations to enhance accuracy. sEEG channels identified as “bad” (e.g., broken, excessively noisy, or containing artifacts) by clinicians were excluded from the analysis, leaving an average of 95±32 (mean+SD) usable sEEG channels per patient.

At 204, the sEEG recordings were segmented into non-overlapping 500 ms windows to facilitate the fitting of dynamical systems models and feature extraction. All data processing and analysis tasks were carried out using MATLAB R2023b (MathWorks, Natick, MA). Models designed to predict surgical outcomes were developed using Python 3.6 (Python Software Foundation, Wilmington, DE).

At 206, dynamical brain network modeling was performed. In general, Dynamical Network Models (DNMs) include generative models that delineate the dynamic influence of individual iEEG channels across the iEEG network. An interictal DNM adopts a Linear Time-Varying (LTV) framework to model interactions between observed brain regions (iEEG channel signals). The LTV model may include Linear Time-Invariant (LTI) DNMs, each based on non-overlapping windows of duration T. The study selected T=500 ms. The LTI model used in the study for each interval included the equation

x(t+1)=Ax(t),

where x(t) ∈ [symbol] ^{N} symbolizes the iEEG channels and A ∈ [symbol] ^{N×N} is the state transition matrix that captures channel interactions. For the study, the transition matrix A for the 500 ms window was fit through a least-squares optimization, by way of non-limiting example.

At 208, the sink indices were computed from the DNMs. In iEEG dynamic networks (DNMs), nodes—channels representing brain regions—may be categorized as sources or sinks according to their relative influence within the network, as represented by A. Sources may be characterized by their significant influence on other nodes while being minimally affected by them. Conversely, sinks may be characterized by their low influence on other nodes but high susceptibility to influence from the network. The influence of each node may be quantified by summing the absolute values of A along the rows

$$( {r}_{it} {\sum }_{j = 1}^{N} \vert {A}_{i j} \vert )$$

and columns

$$( {c}_{it} {\sum }_{j = 1}^{N} \vert {A}_{j i} \vert ) ,$$

where N is the number of nodes. To account for differences between channels, both the row and column sums are subjected to rank transformation, resulting in the row rank (rrᵢₜ) and column rank (crᵢₜ), respectively. The sink index, vᵢₜ, may be subsequently defined as, by way of non-limiting example:

$${v}_{it} = \sqrt{2} - \sqrt{{( 1 - r {r}_{it} )}^{2} + {( \frac{1}{N} - c {r}_{it} )}^{2}}$$

A higher sink index indicates a greater tendency of a node to act as a sink, whereas a lower sink index suggests a stronger inclination towards being a source. For the study, an optional heatmap was used to visualize variations in sink indices.

At 210, SISE was computed from the sink indices. Note that although the study used SISE, embodiments may use any assessment of the entropy of a frequency spectrum of tendencies to act as a sink. In general, an entropy may quantify a lack of order, and thus spectral entropy may assess the complexity or randomness of a signal within the frequency domain. Spectral entropy serves as a tool to ascertain the signal's regularity by examining its frequency distribution. The overall assessment used by an embodiment may quantify the lack of order among the frequency spectrum over the iEEG channels of a tendency to act as a network sink.

The study computed the SISE of each channel, denoted SISEᵢ, by first computing the power spectral density (PSD) of the sink index signal (vᵢₜ), denoted Pᵢ[k], in an overlapping sliding window of 1 min (120 samples) with a step size of 1 sample, using fast Fourier transform. The PSD signal Pᵢ[k] determines the power present at each frequency k. This PSD was then normalized to create a probability density function (PDF), P̅ᵢ[k], ensuring the total sums to one. The study assessed SISE by applying Shannon's entropy formula to this normalized PSD, essentially summing the product of each frequency's normalized power and the logarithm of this power as follows, by way of non-limiting example:

$${SISE}_{i} = - {\sum }_{k = 0}^{N - 1} {\overline{P}}_{i} [ k ] {log}_{2} {\overline{P}}_{i} [ k ]$$

The result is a measure of the unpredictability or evenness in the frequency distribution. A signal characterized by a uniform spread of power across frequencies exhibits higher spectral entropy, indicating a less predictable and more complex structure. Conversely, a signal that displays a narrow distribution of dominant frequencies will have a lower spectral entropy, suggesting more predictability and less randomness.

At 212, the average SISE signal is monitored for significant drops. For each channel, by way of non-limiting examples, the area (AUT) and duration (TUT) below the threshold were quantified, providing a feature representation of each seizure event. Thresholds were applied to the average SISE of the network, which may be represented as follows, by way of non-limiting example:

$$\overline{SISE} = \frac{1}{N} {\sum }_{n = 0}^{N - 1} {SISE}_{n}$$

The threshold was determined on an individual patient basis to minimize the total error that included both false negative and false positive detections. Amongst others, the threshold may depend on the variance of the noise. More specifically, the threshold may be calculated by kσ(S̅I̅S̅E̅), where k is set individually for each patient.

To select an optimal patient-specific threshold, the study analyzed the long-term data from the EMU and chose k to be equal to 6 (by way of non-limiting example). This value minimized the total error rate, balancing both false negatives and false positives. It was observed that setting k=6 consistently yielded the lowest total detection error, thereby optimizing the sensitivity and specificity of the seizure detection system for individual patients. This approach ensured that the threshold adapted to the unique noise characteristics and seizure patterns of each patient, providing a tailored solution for accurate seizure monitoring.

Following the detection of a SISE drop, the study quantified the extent of this decrease for each channel using two metrics: Time Under Threshold (TUT) and Area Under Threshold (AUT). TUT, measured in seconds, quantifies the duration of the entropy reduction for a channel during an event. AUT, expressed in entropy units ranging from 0 to 1, quantifies the magnitude of the entropy reduction for a channel during an event. To integrate these metrics, the study introduce the Event Activity Metric (R score), which combines AUT and TUT through the formula R=TUT/(0.6-AUT). In the formula, an increase in both TUT and AUT resulted in a higher value. This is hypothesized to correlate with seizure activity exhibited by channels, deviating from their interictal baseline. The denominator was experimentally set to 0.6-AUT based on observed data.

Capturing AUT, TUT, and computing the R score across all brain channels yielded three features per channel per detected event. Each event may thus be represented as a vector with 3N dimensions, where N is the channel count. For datasets with more than two seizures, the study used the Principle Component Analysis (PCA) to reduce dimensions from 3N to 2. This process groups seizures with similar SISE drop characteristics closer together in a reduced dimensional space.

To determine whether a particular channel is within the SOZ, the study analyzed the SISE drops. By computing specific metrics, the SOZ may be accurately localized. For all seizures classified under the same type Ci, the study computed the average channel's SISE across the seizure period, which may be represented as follows, by way of non-limiting example:

$${\overline{SISE}}_{i}^{( {C}_{k} )} = \frac{1}{{M}_{k}} {\sum }_{j ∈ {C}_{k}} {SISE}_{i} ( j )$$

In the above equation, Mₖ is the number of seizures in the same group (Cₖ), and S̅I̅S̅E̅ᵢ^{(CK)} is the average SISE for a channel across all seizures in the group.

After averaging these waveforms to compute S̅I̅S̅E̅ᵢ^{(Ck)} for each channel i, the study then computed AUT, TUT, and the R score (Ri) for each channel i. Channels that show a consistent and significant decrease in entropy across a majority of seizures of a particular type belong to the SOZ.

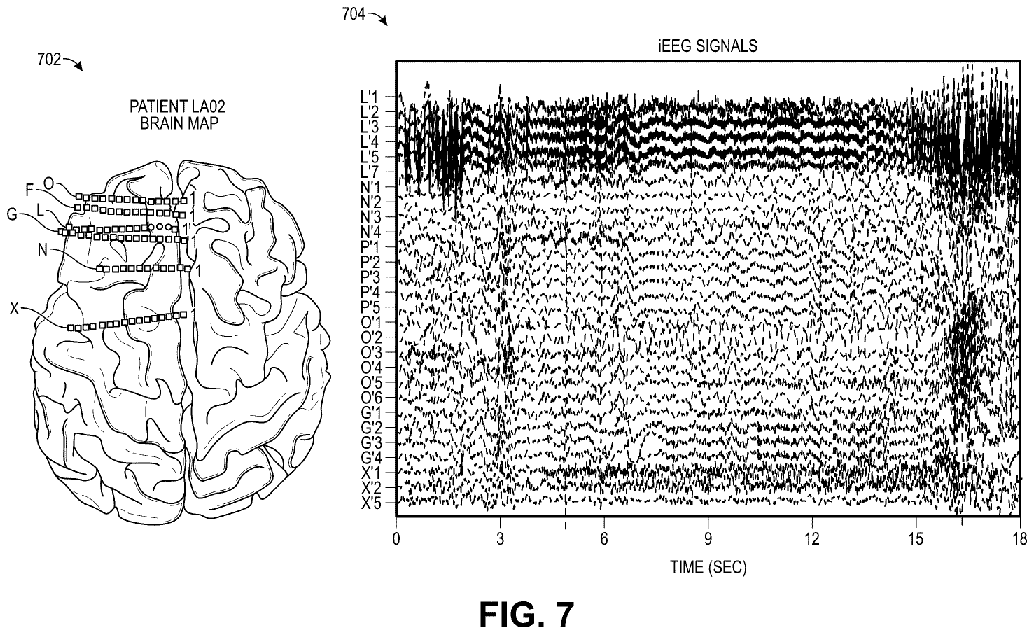

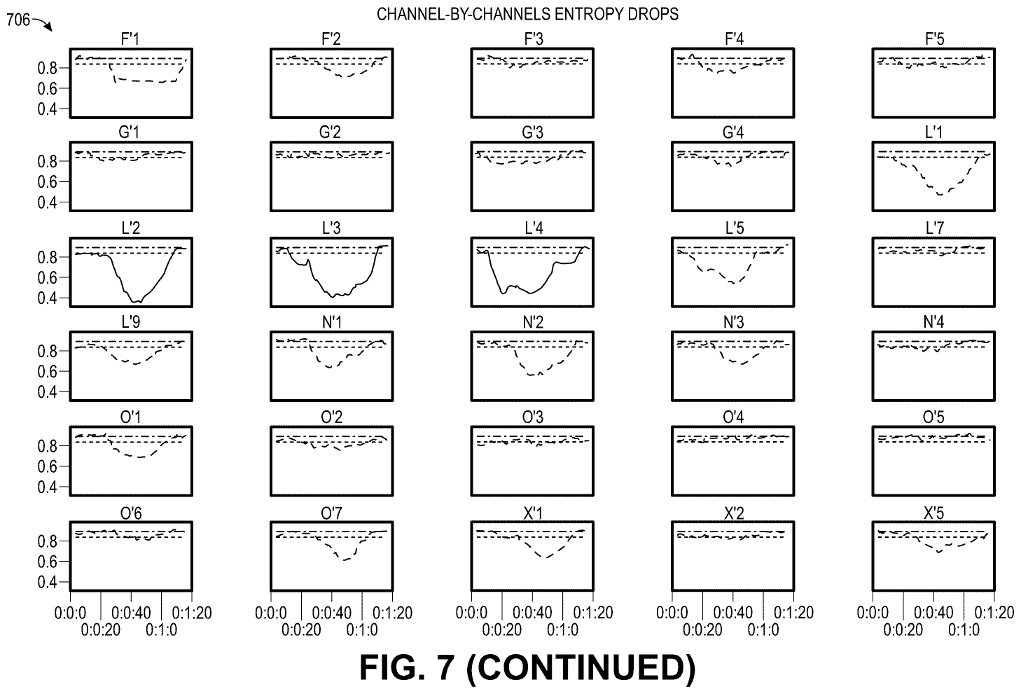

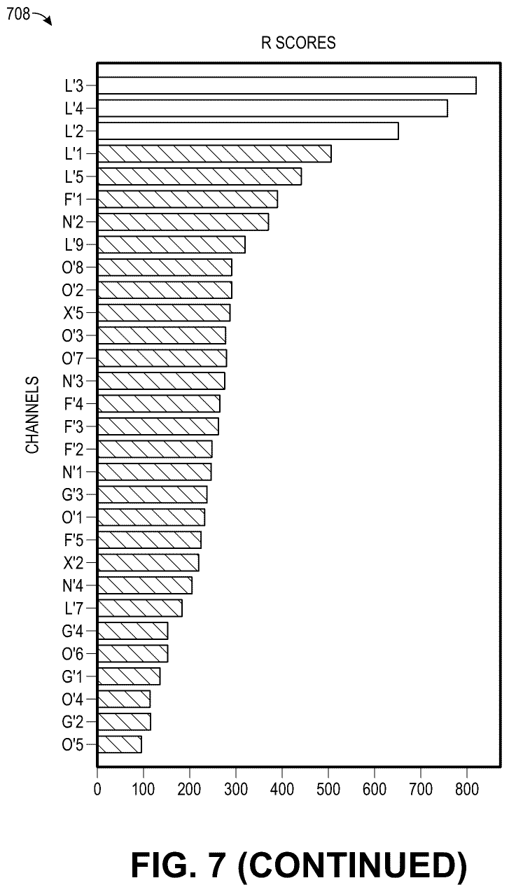

As noted, the study evaluated the R score for SOZ localization. In particular, the study evaluated the performance of the metric on patients with successful outcomes. To assess the effectiveness of the R score for SOZ localization, the study determined the optimal threshold and used leave-one-out cross-validation (LOOCV). Each round of cross-validation involved partitioning patients into two complementary subsets: train and test sets. The train set included the R scores of channels from all successful patients except one, while the test set included the R score of the single excluded patient. The study then computed the ROC curve on the test data to identify an optimal threshold for distinguishing SOZ channels from non-SOZ channels. Using this threshold, the study evaluated the patients in the test set. Specifically, the independent variable in training the classifier was the R score of each channel, and the dependent variable was a Boolean indicating whether the channel was in the SOZ (0 for non-SOZ, 1 for SOZ). This process was repeated iteratively for all patients. Further discussion of using the R score for SOZ localization, including the results of the aforementioned analyses, appears in reference to FIG. 7, below.

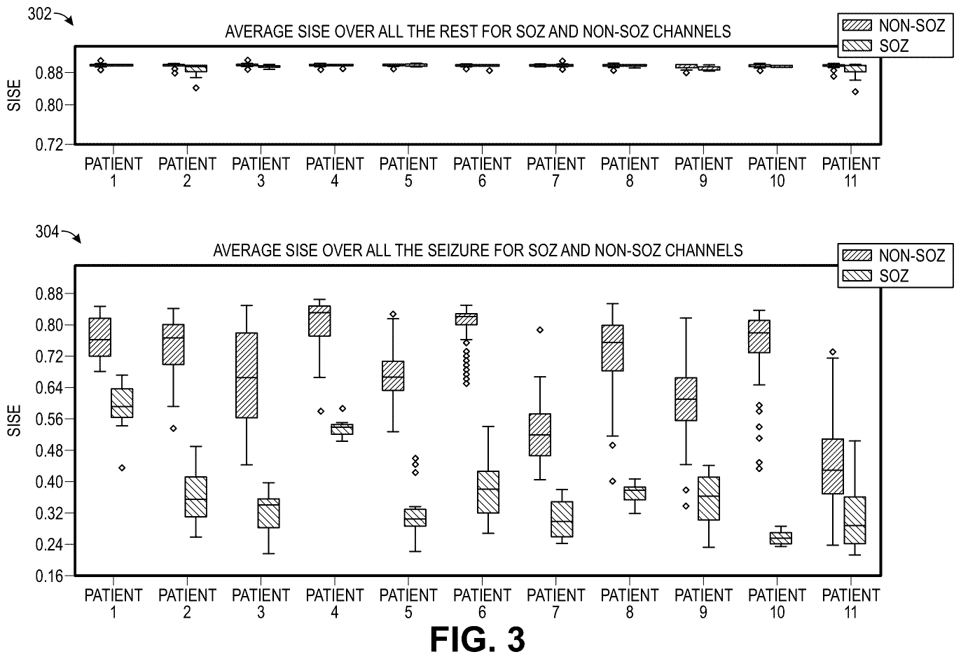

FIG. 3 depicts a comparative analysis of SISE values in SOZ and Non-SOZ channels during interictal (rest) and seizure phases, according to the non-limiting example embodiment of the study.

In particular, FIG. 3 shows a comparison 302 of SISE values during the interictal (rest) period for SOZ and Non-SOZ channels for each patient. Boxes represent the distribution of channels' SISE mean values in 15-second segments, averaged over the entire interictal period of inpatient epilepsy monitoring. FIG. 3 also shows a comparison 304 of SISE values during seizure periods for SOZ and Non-SOZ channels for each patient. Boxes represent the distribution of channels' SISE mean values in 15-second segments, averaged over all seizure periods captured during inpatient epilepsy monitoring.

The study computed the SISE signal for the entire duration of in-patient monitoring for each of the 11 patients in the long-term cohort. The results illustrated in FIG. 3 show that the average SISE across all iEEG channels and across patients is significantly lower during seizure periods compared to interictal periods (comparison 302). This indicates a significant drop in network variability over small time windows during seizures. During seizure periods, channels in the clinically annotated SOZ exhibit significantly lower SISE values compared to non-SOZ channels (comparison 302). In contrast, during non-seizure periods, this difference between SOZ and non-SOZ is not significant (comparison 304).

The study also evaluated the utility of the SISE drop for seizure detection by analyzing data from the 54 patients in the second cohort who underwent in-patient continuous iEEG monitoring. The study computed the SISE scores for all patients and found significant reductions in average SISE values compared to baseline across the brain network during seizures. This drop indicates a substantial decrease in the spectral complexity and variability of the average source/sink activity within the brain's network, reflecting the localized synchronization typical of seizure states.

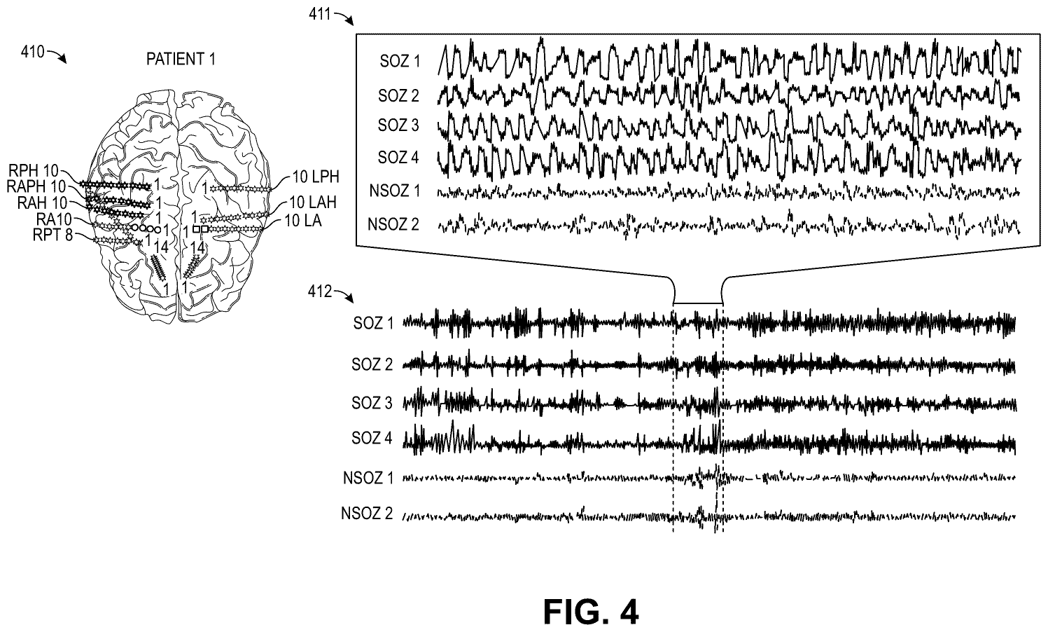

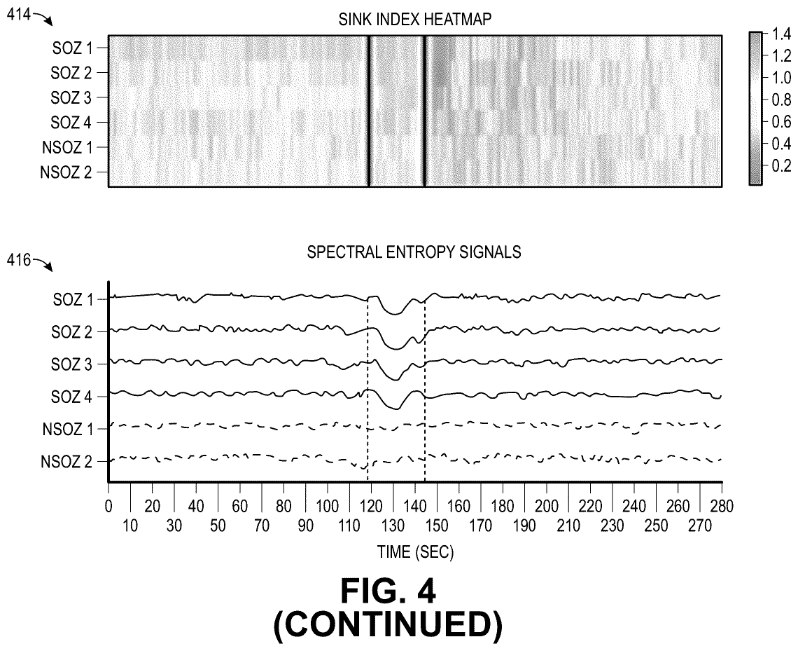

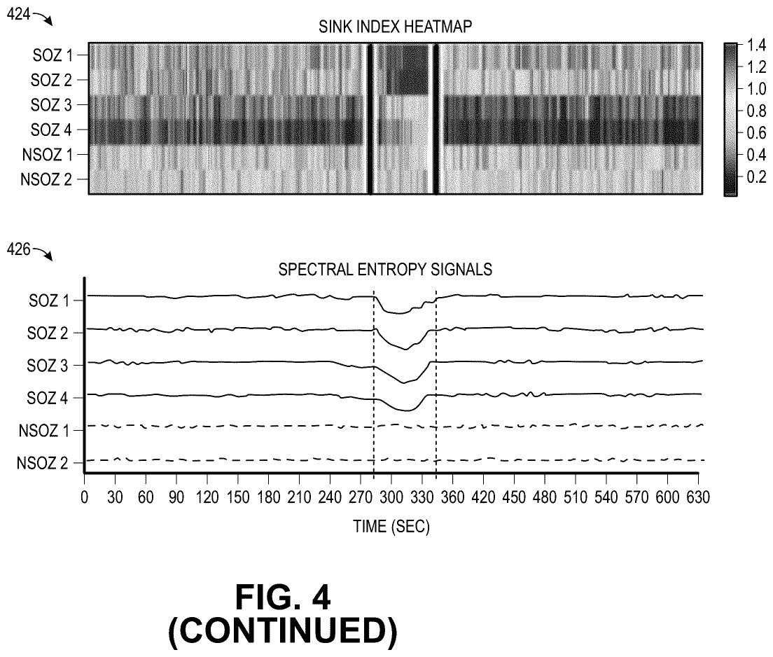

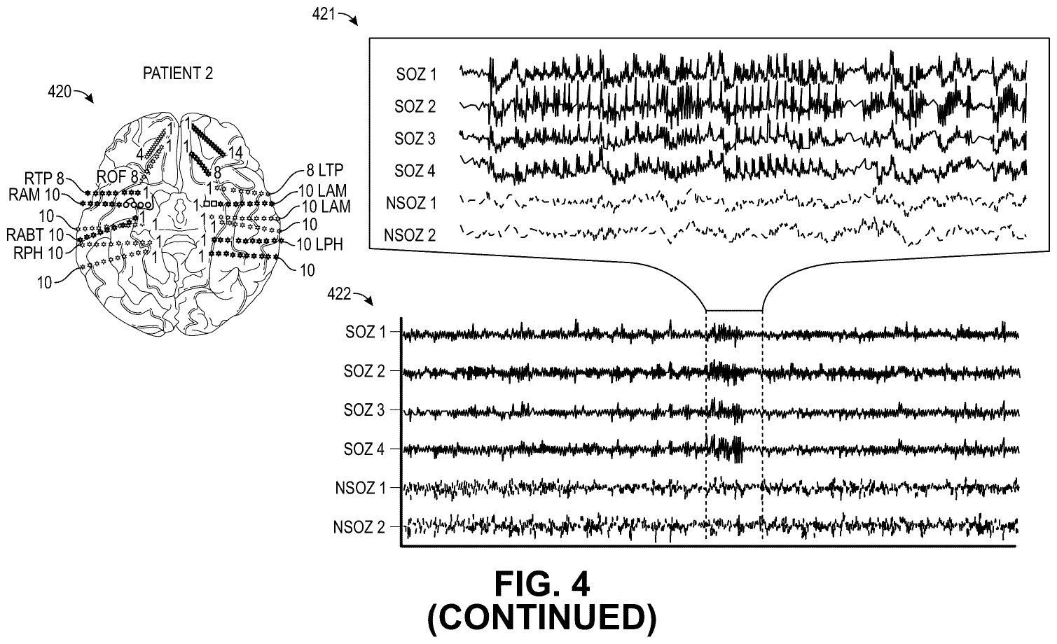

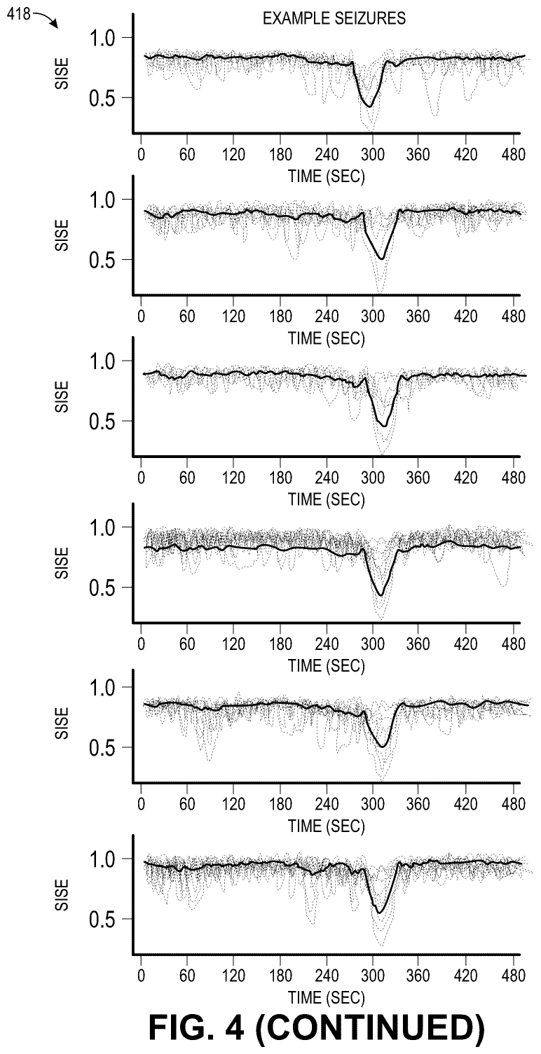

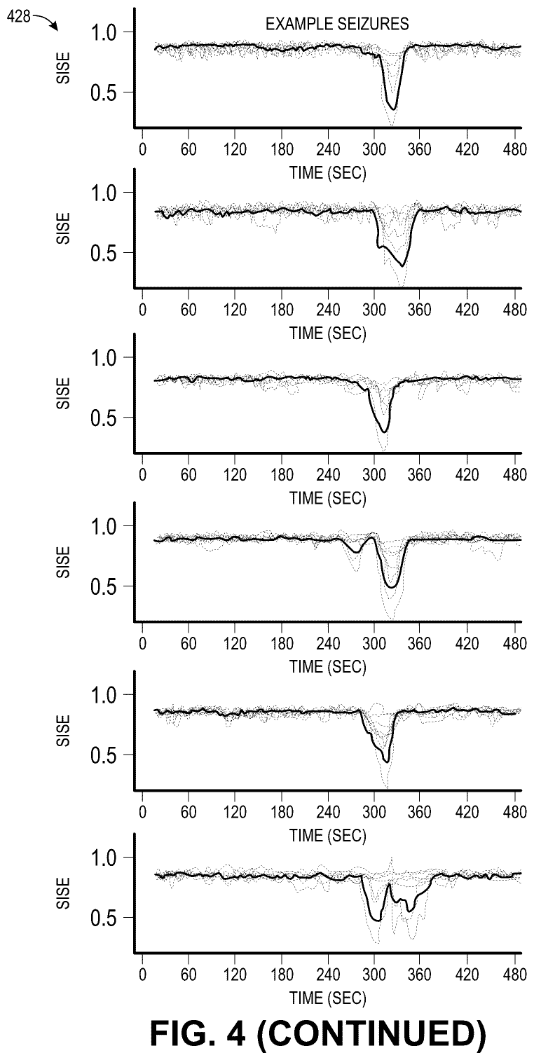

FIG. 4 illustrates iEEG signals 412, 422 from multiple channels for two example patients according to the non-limiting example embodiment of the study. FIG. 4 also shows SISE signals 416, 426 and corresponding sink index heatmaps 414, 424. FIG. 4 further shows MRI-reconstructed brain images 410, 420 of the two patients, with corresponding implantation maps. The iEEG signals 412, 422 for the patients, with different electrodes on the y-axis, are shown four minutes before and after a clinician-annotated seizure. Vertical lines bound the time of the seizure. Solid traces indicate contacts in the SOZ, and dashed traces mark Non-SOZ (NSOZ) contacts. Each iEEG signal 412, 422 is scaled for visualization to a range from 200 μV to 2,000 μV. The sink index heatmaps 414, 424 are for a subset of channels within the clinically annotated SOZ. FIG. 4 shows SISE traces for all channels (thin dotted traces) and the channel average (solid trace) from example seizures. The channel averages exhibit a significant deviation from the baseline at the seizure onset.

FIG. 4 further includes exploded views 411, 421 of the sink index entropy over one-minute windows for SOZ (solid traces) and NSOZ (dahsed traces) contacts. Clinicians observed high-frequency, synchronized spiking activity at the seizure onset (solid traces). The sink indices for these electrodes (visualized by the heat maps 414, 424) change and lose temporal variability precisely at seizure onset. This sudden change and loss of the natural stochastic variability in these subset of channels manifests as a gradual loss of SISE on those channels (416, 426, solid traces). Conversely, other channels that were not involved in the seizure, or were less involved, exhibited a smaller drop in SISE (416, 426, dashed traces). Consequently, average SISE values over the brain network consistently drop across all seizures (418, 428).

The analysis of the study revealed a sensitivity of 0.98 and 1, and a precision of 0.94 and 1, for the 11 patients in the continuous iEEG cohort and 43 patients in the seizure snapshot cohort, respectively. Results for patients in continuous iEEG cohort are presented in Table 1. Most patients had sensitivity and precision levels well above 0.90. The SISE seizure detector demonstrated low false positive (FP) and false negative (FN) rates. The average F1-score was 0.96, which indicates a strong balance between true seizure detection and avoiding false positives.

TABLE 1

| | CR | NCR | | | | | |
|---|---|---|---|---|---|---|---|
| Patient | TP | TP | FP | FN | Sensitivity | Precision | Accuracy |
| 1 | 3 | 0 | 0 | 0 | 1 | 1 | 1 |
| 2 | 18 | 4 | 2 | 1 | 0.96 | 0.92 | 0.92 |
| 3 | 25 | 23 | 6 | 0 | 1 | 0.89 | 0.89 |
| 4 | 1 | 0 | 0 | 0 | 1 | 1 | 1 |
| 5 | 47 | 2 | 0 | 0 | 1 | 1 | 1 |
| 6 | 47 | 12 | 6 | 2 | 0.97 | 0.91 | 0.94 |
| 7 | 55 | 22 | 2 | 0 | 1 | 0.97 | 0.98 |
| 8 | 23 | 14 | 0 | 1 | 0.97 | 1 | 1 |
| 9 | 31 | 34 | 2 | 0 | 1 | 0.97 | 0.97 |
| 10 | 29 | 7 | 9 | 3 | 0.92 | 0.80 | 0.81 |
| 11 | 12 | 0 | 0 | 0 | 1 | 1 | 1 |

Table 1 presents performance metrics of the SISE seizure detector across 11 patients in the continuous iEEG cohort. The table shows the number of true positives for clinically-reported seizures (CR TP) and non-clinically-reported (NCR TP), false positives (FP), false negatives (FN), sensitivity, precision, and accuracy for each patient.

The non-limiting embodiment of the study identified significant seizure events not labeled during routine monitoring. Overall, from 409 seizures identified in 11 patients, 118 were later confirmed to be true seizures not clinically annotated, increasing seizure detection accuracy in invasive epilepsy monitoring by 135%+36% on average for each patient.

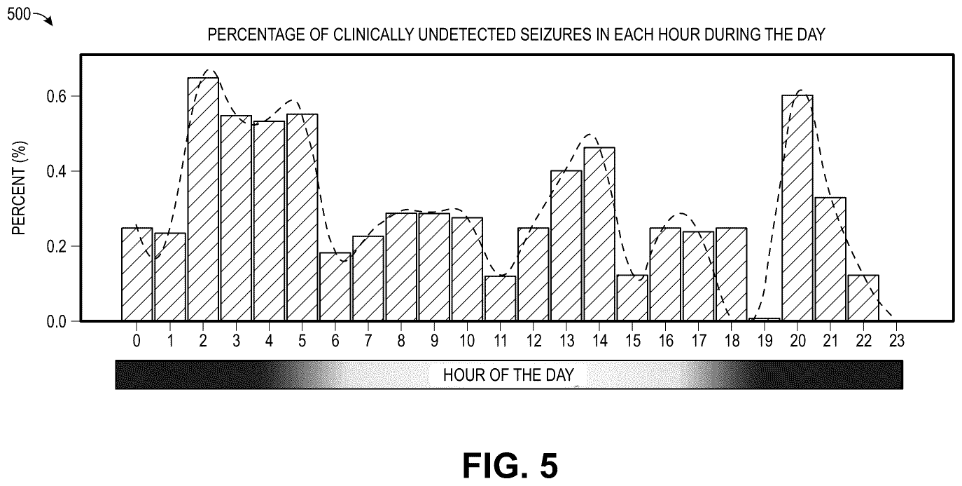

FIG. 5 shows a distribution 500 of clinically undetected seizures by hour of the day. The x-axis represents the hour of the day, ranging from 0 (midnight) to 23 (11:00 PM). The y-axis represents the percentage of seizures that went clinically undetected, expressed as a fraction (0.0 to 0.4). As shown in FIG. 5, the highest percentage of undetected seizures occurred around 02:00 (2:00 AM) and 07:00 (7:00 AM) and also around 20:00 (8:00 PM). Possible reasons for these increased percentages include changes in clinical staff shifts, variations in monitoring intensity during these times, the lack of generalizability of current seizure detectors to new patients, and insufficient manpower for continuous manual seizure detection throughout the day. Based on these findings, an embodiment can enhance iEEG seizure detection in clinical settings, especially for managing large volumes of iEEG data where subtle seizure activities might otherwise go unnoticed.

The study also determined that quantifying SISE drop across channels can facilitate the identification of seizure types. While the average SISE analysis allows for accurately detecting seizures, the averaged SISE signal lacks granularity that may help elucidate the specific brain areas involved and their extent. Therefore, the study explored the contribution of per-channel SISE values to the overall average SISE signal.

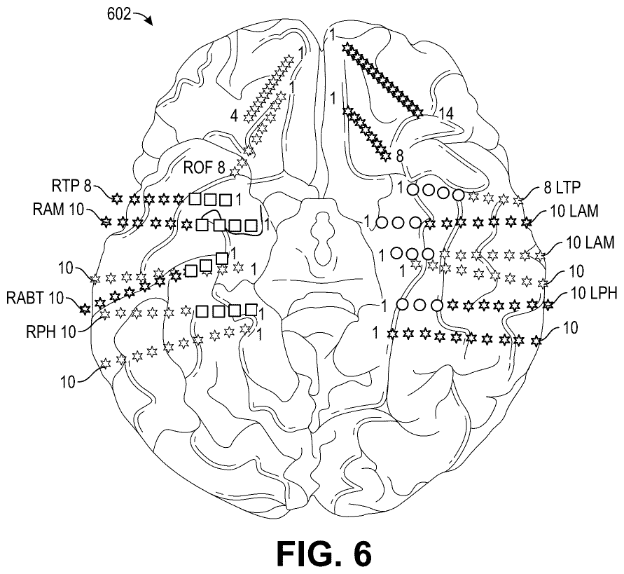

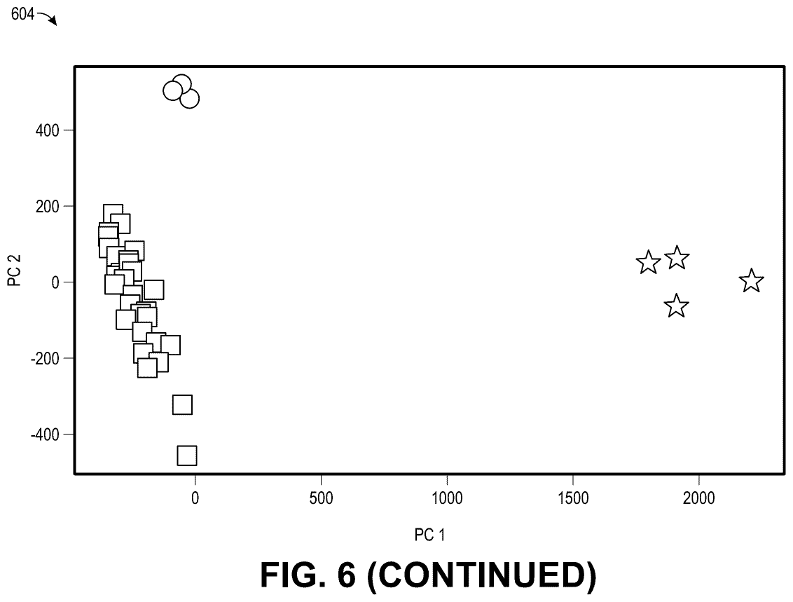

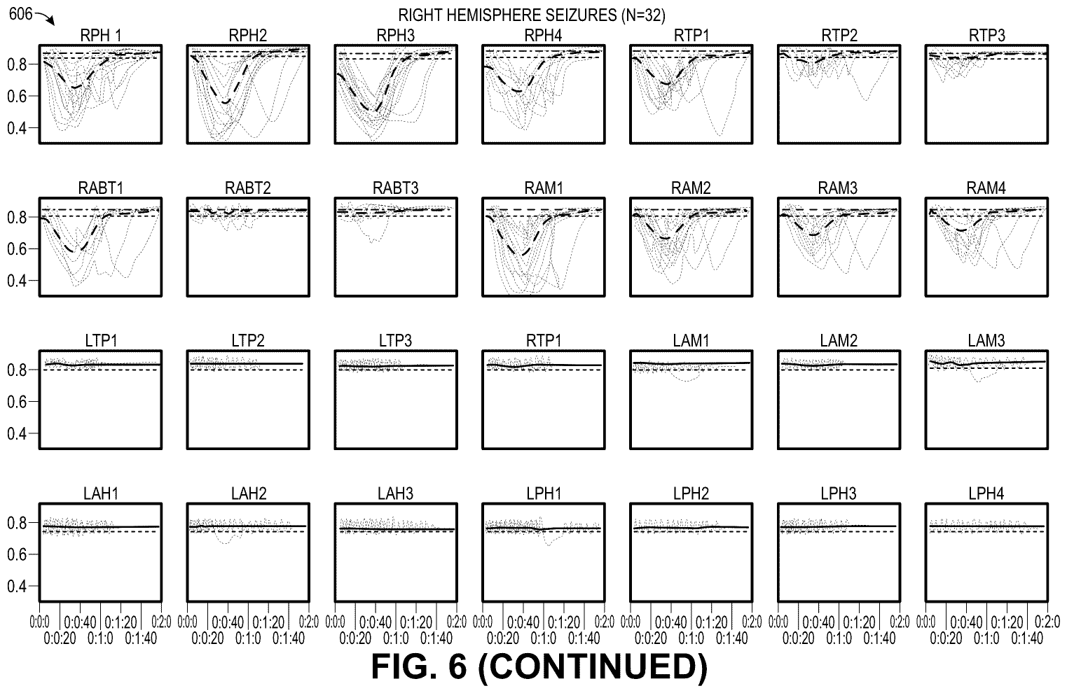

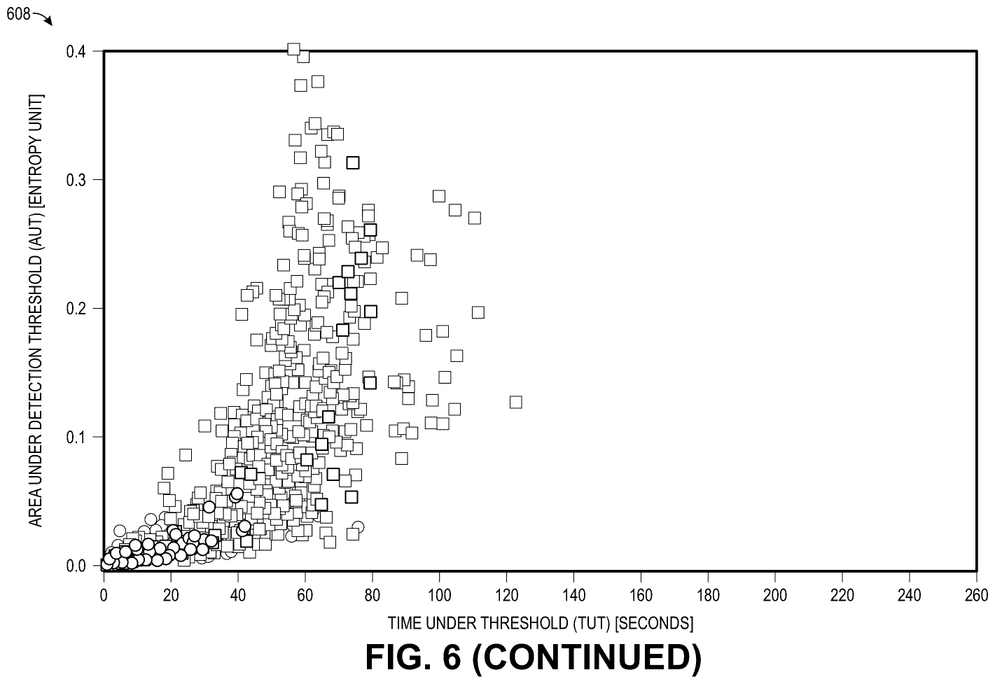

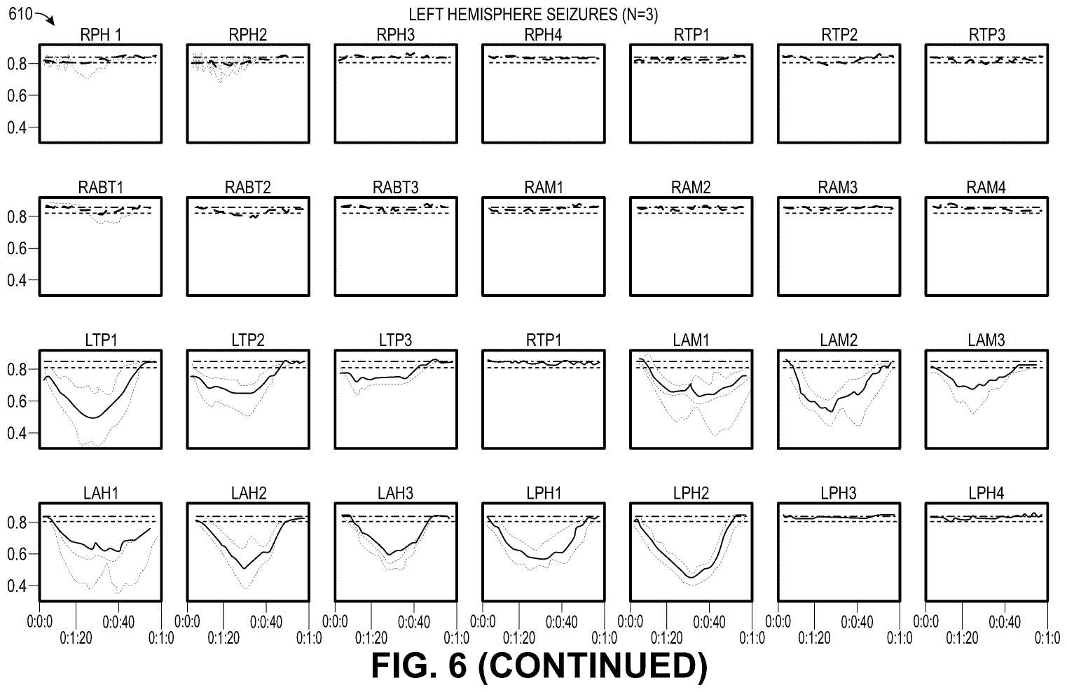

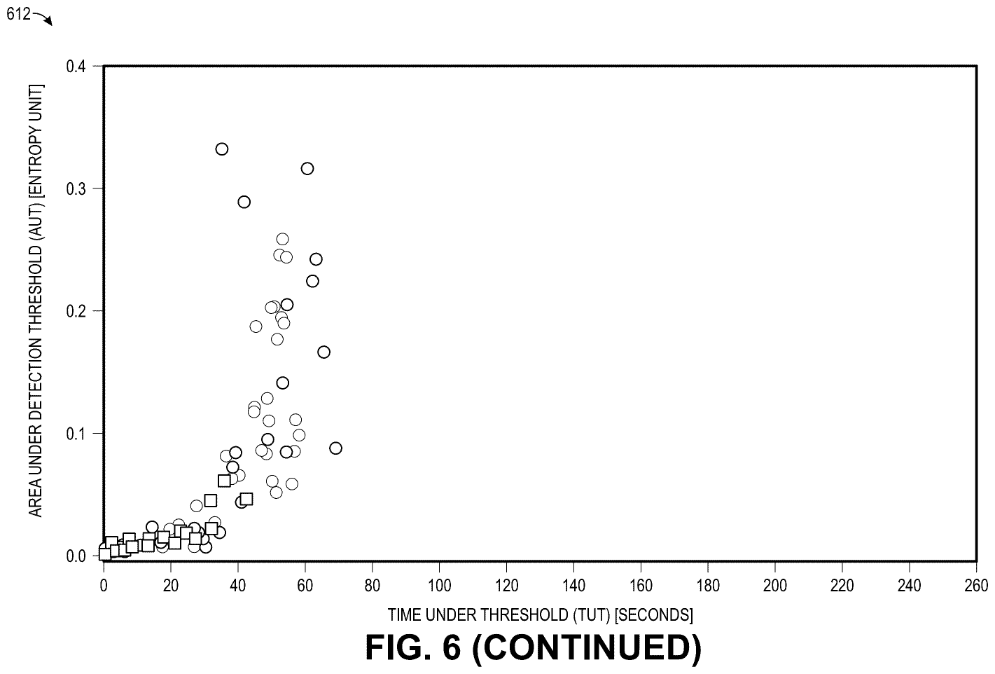

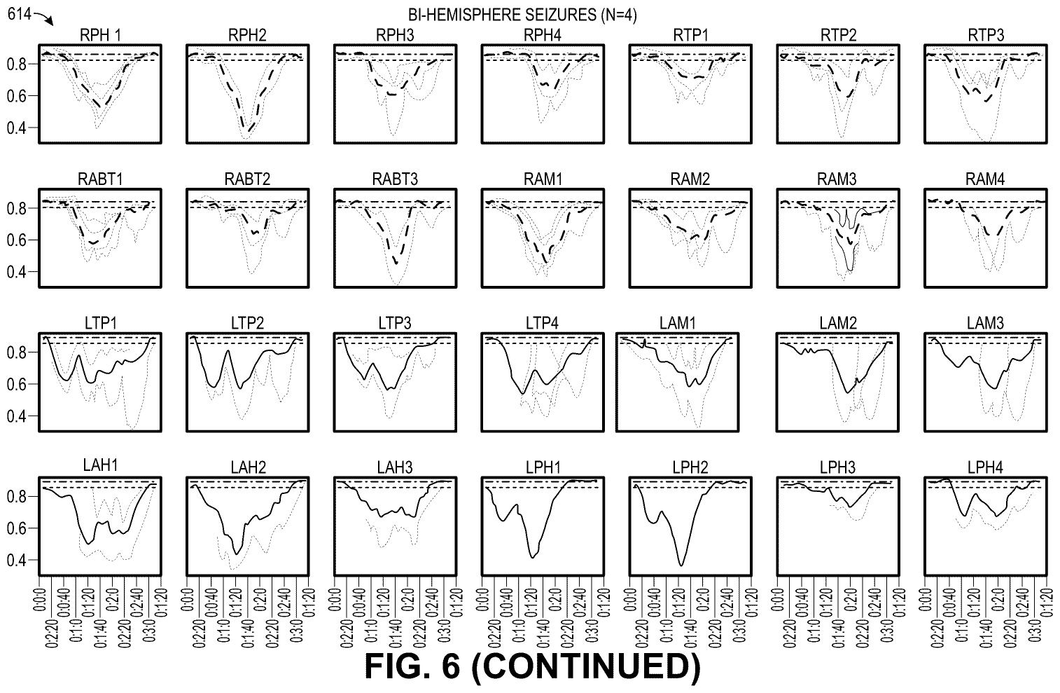

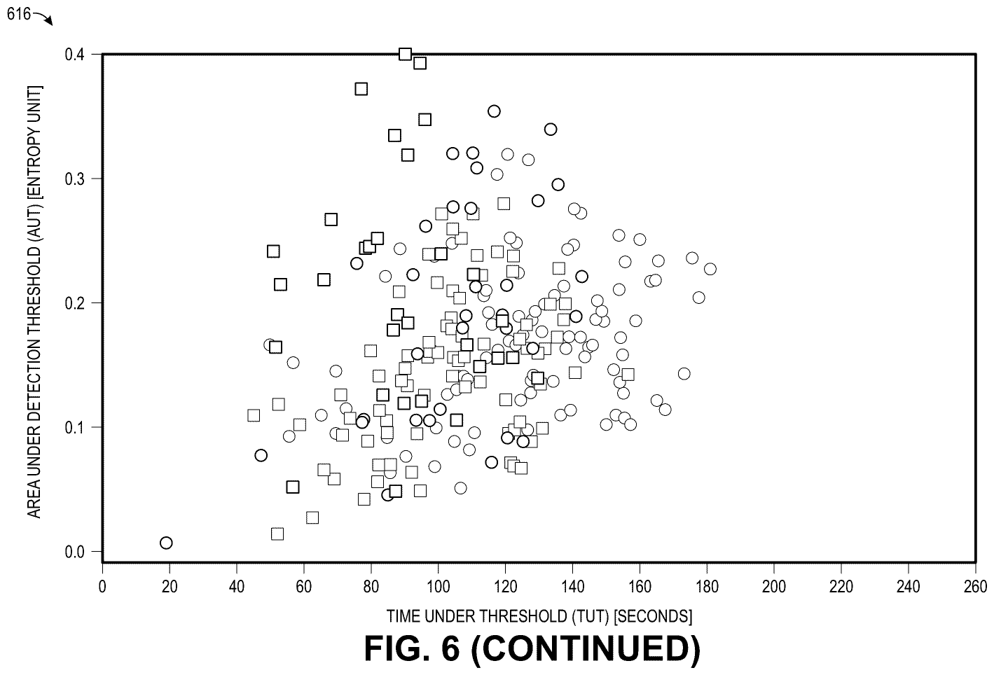

The study observed that during average SISE drops, not all channels exhibit similar reductions. Different seizures involve different sets of channels driving the drop in the averaged signal. This variability indicates the potential for multiple seizure types per patient. For example, in one particular patient, the study identified multiple distinct profiles of SISE drops across brain areas during different seizure events, as shown and described in reference to FIG. 6. In particular, using PCA dimensionality reduction, the study identified multiple seizure profiles for this patient.

FIG. 6 illustrates distinct seizure clusters in low-dimensional space and their corresponding seizure profiles for a particular patient in the study. FIG. 6 shows an MRI-reconstructed brain map 602 of a DRE patient undergoing fifteen-day iEEG monitoring. Seizures arise from either and both hemispheres, necessitating bi-hemispheric implantation. As shown in FIG. 6, the reduced dimensional (2D) PCA scatter plot 604 of the SISE values shows distinct clusters, with circles indicating left hemisphere and squares indicating right hemisphere. FIG. 6 further shows channel-by-channel SISE scores 606 for channels in the right (dashed traces) and left (solid traces) hemispheres, which indicate a cluster of right hemisphere seizures. The scatter plot 608 of each channel's SISE dip features (time and area under threshold) highlight the channels most involved for the right hemisphere seizures. Likewise, the channel-by-channel SISE scores 610 for channels in the right (dashed traces) and left (solid traces) hemispheres indicate a cluster of left hemisphere seizures, and the corresponding scatter plot 612 highlights the channels involved for the left hemisphere seizures. Finally, the channel-by-channel SISE scores 614 for channels in the right (dashed traces) and left (solid traces) hemispheres indicate a cluster of bi-hemisphere seizures, and the corresponding scatter plot 616 highlights the channels involved for the bi-hemisphere seizures.

In the particular patient whose data is illustrated in FIG. 6, 39 seizures were detected and validated using the SISE seizure detector, as shown in the PCA scatter plot 604. Each seizure is represented as a circle (left hemisphere) or square (right hemisphere) in the 2D PCA-space. The analysis reveals three clusters of seizures with distinct profiles. Channel-wise SISE signals averaged over all seizures within each cluster showed varying patterns: focal seizures in the right hemisphere (SISE scores 606), focal seizures in the left hemisphere (SISE scores 610), and generalized seizures involving both hemispheres (SISE scores 614). More specifically, the first cluster (squares) included SISE drops only in the right hemisphere, the second cluster (circles) included SISE drops only in the left hemisphere, and the third cluster (stars) had SISE drops across both hemispheres. The TUT, AUT scatter plots (608, 612, 616) provides further visualization of these clusters, displaying each channel as a symbol (circle, square, or star), with one seizure in each cluster shown in higher opacity for better clarity.

Thus, the technique of the study provides a quantitative and interpretable method of classifying seizures into different types, which enhances physician understanding of the underlying epileptic network. By identifying distinct seizure profiles and their corresponding brain areas, clinicians can tailor treatment strategies more effectively, improving patient outcomes and advancing the management of epilepsy.

Embodiments provide a reliable biomarker for SOZ localization. Note that beyond classifying different seizure types, identifying the exact brain areas involved during epileptic events, i.e., localizing the SOZ, is important to proceeding with surgical interventions. To explore the relationship between channel-by-channel SISE R scores and periods of seizure activity, the study compared the SISE values for different channels before, during, and after a seizure. The study observed that the SISE signal for channels in the clinically-annotated SOZ displayed a much larger drop compared to channels outside the SOZ (FIG. 4, reference 416, 426, solid and dashed traces, respectively, for two example patients). This trend generalizes over multiple seizures (FIG. 4, references 418, 428), where the average SISE across all channels (FIG. 4, references 418, 428, solid curve) is driven by only a subset of channels (FIG. 4, solid traces). The channels exhibiting the earliest and largest SISE drops drive the seizure events and represent the SOZ.

To investigate whether the SISE drop can accurately localize the SOZ, the study considered patients who underwent successful epilepsy surgery that resected the clinically determined SOZ. Specifically, the study examined if regions whose resection led to seizure-free outcomes exhibited a more pronounced SISE drop. As an example, FIG. 7 represents one patient who was seizure-free after surgery.

FIG. 7 illustrates SOZ localization using an SISE R score according to the non-limiting example embodiment of the study. FIG. 7 shows MRI-reconstructed brain images 702 of a DRE patient undergoing invasive iEEG monitoring with implanted electrode locations overlaid. Circles indicate channels within the clinically annotated SOZ that were later successfully resected with a seizure-free outcome. FIG. 7 also shows raw iEEG traces 704 for one seizure from the patient, with electrodes labeled on the y-axis. Solid traces correspond to the clinically annotated SOZ contacts. All traces are scaled to 200 μV to 2,000 V for optimal visualization. FIG. 7 also shows corresponding channel-wise SISE signals 706. Finally, FIG. 7 shows SISE R scores 708 of entropy drop for each channel during the seizures. Channels with higher SISE R scores (white bars) match the clinician-labeled and resected areas.

For the patient who was seizure-free after surgery represented by FIG. 7, the channel-by-channel SISE drops for the seizure illustrated by the EEG traces 704 clearly exhibited a greater entropy drop (SISE signals 706) and a higher SISE R score (SISE R scores 708) in the clinically annotated SOZ as compared to non-SOZ channels. The clear differentiation between these channels underscores the effectiveness of SISE, and more generally an assessment of the entropy of a spectrum of tendencies to act as a sink, in identifying an SOZ.

To evaluate the general overall effectiveness of the non-limiting example embodiment, the study analyzed data from 43 patients across 5 centers. First, the distributions of SISE R score drops between the clinically annotated SOZ and non-SOZ (NSOZ) channels were computed. Data from all patients was pooled into two groups: one who had successful surgical outcomes and one who had post-resection seizures, indicating a failed epilepsy surgery. The predicted SOZ, estimated using the SISE R score distributions, was compared to the clinically annotated SOZ used for the resections. For the group with successful outcomes, there was a high correspondence between the SISE SOZ and the resected channels. For the group with unsuccessful outcomes, the SISE SOZ and resected channel labels correspondence was significantly lower, suggesting that either (1) areas that were potentially part of the SOZ were not resected, or (2) the depth electrodes were inserted in regions further away from the true SOZ. To explore these options, the study compared the SISE R scores between the resected and non-resected channels within each group and noted a significantly greater overlap in the scores for the unsuccessful cases. Thus, it is likely not the case that the SOZ was mislabeled; rather it is likely that for these patients, either the SOZ was less prominent or that the SOZ was not near the implanted channels.

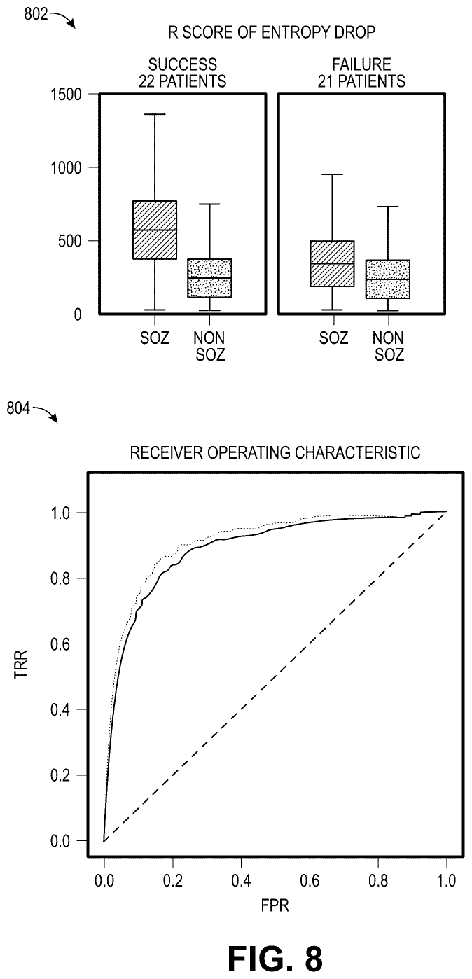

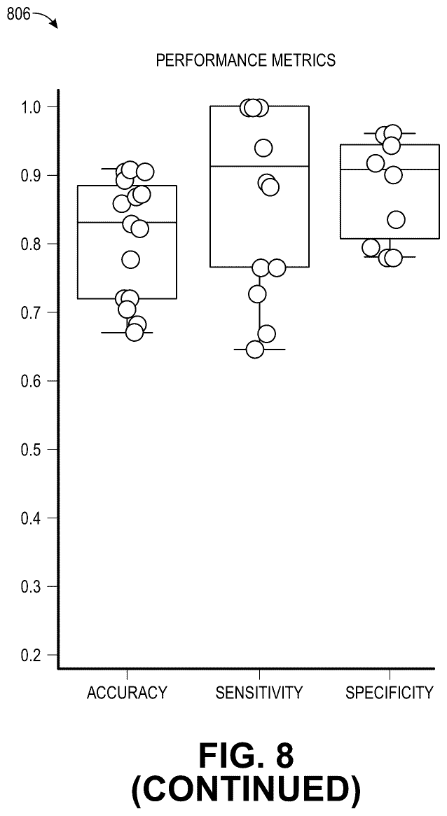

As noted above in reference to FIG. 2, the study also performed a classification experiment using randomized leave-one-out cross-validation (LOOCV) to find the optimal threshold for classifying SOZ from NSOZ channels. This classification used the SISE R score to determine the robustness of the SISE drop feature. Performance metrics for this analysis are presented in reference to FIG. 8.

FIG. 8 illustrates seizure onset localization performance metrics 806 as evaluated for the study. FIG. 8 shows the SISE R scores 802 for 22 patients with successful surgical outcomes and 21 patients without successful surgical outcomes. The R scores are compared between the SOZ and non-SOZ regions. In the successful outcome group, the SOZ shows higher R scores compared to non-SOZ regions, with the distributions being essentially disjoint. In the unsuccessful outcome group, the R scores are lower and similar between SOZ and non-SOZ regions. FIG. 8 also presents a Receiver Operating Characteristic (ROC) 804 curve showing the performance of the SISE seizure detector across all patients with successful surgical outcomes, using LOOCV during training. The ROC curve 804 illustrates the true positive rate (TPR) against the false positive rate (FPR), indicating high detection accuracy with an area under the curve (AUC) close to 1. The optimal threshold for distinguishing seizure/non-seizure according to R score was calculated using the ROC curve 804 on the training portion of each iteration. The optimal threshold that maximized the true positive rate (TPR) and minimized the false positive rate (FPR) across 22 rounds of LOOCV was determined to be 385.8±5.7. The average ROC AUC over all rounds of training on all patients except one was 0.88±0.006. Finally, FIG. 8 illustrates performance metrics 806 including accuracy, sensitivity, and specificity for the SISE seizure detector on test data for patients with successful surgical outcomes. In each iteration, the SISE R score on the test subject was evaluated. The accuracy, sensitivity, and specificity were 0.85±0.1, 0.01±0.07, and 0.91±0.13, respectively. The box plots indicate high median values for all three performance metrics 806, demonstrating robust performance in detecting seizures with high accuracy, sensitivity, and specificity.

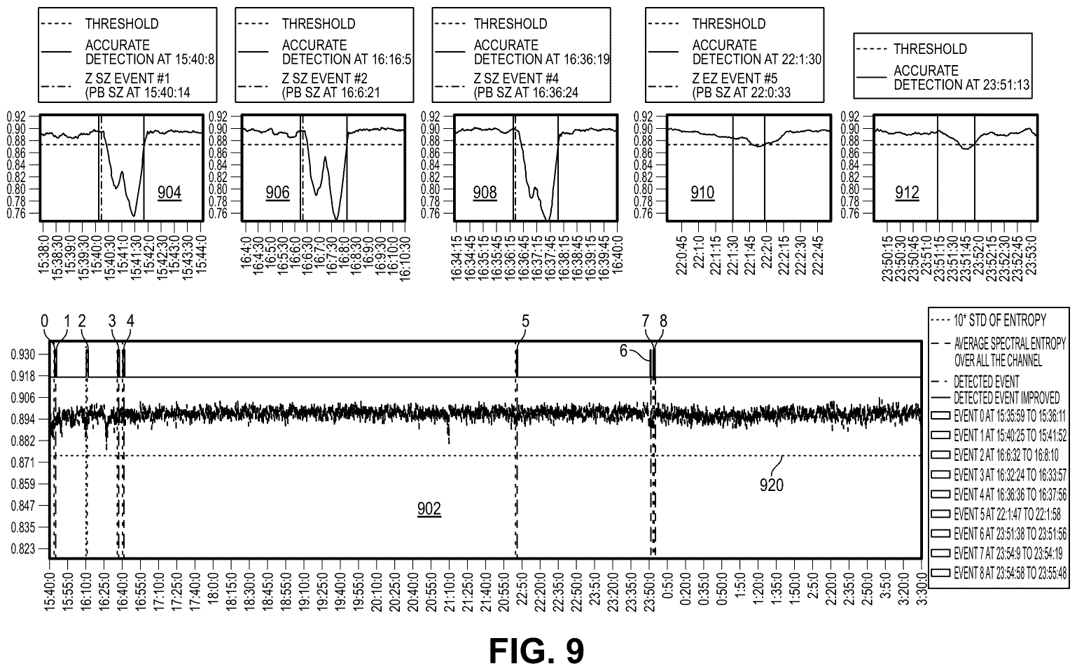

FIG. 9 presents twelve hours of average SISE data 902, computed across all channels in the iEEG recording, as well as detailed SISE average graphs 904, 906, 908, 910, 912 for selected detected events, for a particular patient. In particular, the detailed SISE average graphs 904, 906, 908, 910, and 912 are expanded views corresponding to detected event numbers 1, 2, 4, 5, and 6, respectively. For illustrative purposes, seizure events were detected based on the sum of entropies for all channels, using a threshold 920 of ten times the standard deviation below the mode of sum of channel entropies. Events were detected using a sliding one-minute window, achieving near real-time seizure detection. Note that each of the detailed SISE average graphs 904, 906, 908, 910, 912 is for a different type of seizure, involving different involvement of brain regions, and is unique to each patient.

A discussion of conclusions drawn from the study and a summary of results follow.

During interictal periods, most brain regions do not exhibit strong sink or source behavior. SOZ channels tend to be stronger sinks interictally, with a few functionally connected regions influencing them. However, there is significant variability in the remaining network, with regions often positioned between the extremes of source and sink states. By contrast, during seizure events, the SOZ consistently becomes a group of extremely strong sinks. This likely occurs because several other regions act as sources attempting to correct the aberrant activity in the SOZ. Consequently, the SOZ remains a persistent sink over time, deviating from the typical network dynamics, which lowers the entropy and thus lowers the SISE score used in the study.

This phenomenon is captured effectively by spectral entropy, which reflects the consistent source and sink dynamics indicative of seizures. Seizures exhibit localized synchronization, which can arise either due to local pathological activity patterns within the brain area or via pathological interactions with other brain areas. The analysis of the study identifies how the intra-node influences within the iEEG network become globally less variable during seizures. Specifically, the SOZ emerges as a dominant sink nodes, while non-SOZ regions act as sources. This supports that spatial localization of seizure activity may result from changes in brain-wide interactions.

From a cytological perspective, the source-sink phenomenon is supported by the observed imbalance between elevated levels of glutamate and the inhibitory neurotransmitter GABA in epilepsy. Glutamate, the primary excitatory neurotransmitter in the brain, is a neurotoxic agent, and healthy brain function requires a balance between glutamate uptake and release to maintain extracellular glutamate concentrations within a homeostatic range. Sodium-dependent glutamate transporters (GLTs) are crucial in preventing the accumulation of neurotoxic levels of glutamate in the extracellular space. Increased numbers of GLTs in human dysplastic neurons suggest a protective inhibitory mechanism surrounding the epileptogenic cortex. Thus, the inhibitory (sink phenomena) and excitatory (source phenomena) events within the potential SOZ may have a biological substrate in the differential expression of glutamate transporters within the SOZ.

Beyond seizure detection, embodiments may also classify different seizure types based on channel-by-channel SISE modulations. Identifying distinct seizure profiles allows for an improved understanding of the epileptic network and can inform tailored treatment strategies. Recognizing multiple seizure types in a patient is important for surgical planning, as it may indicate a more complex epileptogenic network that might not be fully addressed by a single surgical intervention. The robustness of the detection presented herein further ensures that all seizure types are more likely to be observed, as some seizure presentations may occur more rarely and therefore be more likely to be missed.

The ability of SISE drops (or other assessments of the entropy of a spectrum of tendencies to act as a sink) to localize the SOZ was validated by comparing SISE R scores of channels in successful and unsuccessful surgical outcomes. Channels with significant SISE drops consistently correlated with regions whose resection led to seizure freedom, demonstrating embodiments' usefulness in guiding surgical decisions.

The SOZ localization of the study was able to set a universal threshold that performed accurately in leave-one-out cross-validation. The accuracy metric used to set the threshold incorporated both false positives and missed detections, selecting a point close to (0,1) on the ROC curve. More generally, these two error modes have very different implications for clinical outcomes. False positives can cause unnecessary resection of unrelated areas, while missed detections can cause SOZ areas to not be resected. The former can induce additional cognitive complications and necessitate additional rehabilitation while the latter can cause the surgery to fail, leading to continued seizure occurrences after the surgery.

The clinical utility of the non-limiting example embodiment of the study, as well as that of embodiments in general, is highlighted by its high accuracy (sensitivity=0.98 and precision=0.94) and ability to detect seizures missed during routine monitoring. These results demonstrate robustness and reliability, making an embodiment a valuable asset in clinical settings, especially for continuous, high-volume iEEG data analysis.

Further benefits of embodiments include detection of seizures that may be missed by human observers. The non-limiting example embodiment of the study detected additional seizures missed during routine clinical monitoring, significantly enhancing the ability of clinicians to analyze very long iEEG recordings with high accuracy. The highest percentage of undetected seizures occurred around 2 AM and 7 AM and around 8 PM (see FIG. 5 and its discussion), likely due to staff shift changes, variations in monitoring intensity, non-generalizable seizure detectors, and insufficient continuous manual detection. To prevent this, EEG technicians and clinicians can use an embodiment to cross-verify suspected seizure events, improving the overall diagnostic process and patient management. Additionally, the automated nature of embodiments can reduce the workload on clinical staff, allowing for more timely and accurate interventions.

Beyond reliability and robustness, the low complexity of embodiments allows for fast computation in real-time settings. Specifically, the non-limiting example embodiment of the study could identify seizures with a maximum delay of 16 s, without needing high-end computing. This capability allows for deployment of embodiments for real-time monitoring of patients in the clinic, facilitating interventions, aid, or generally calling attention to patients for more rigorous monitoring of semiology of seizure. Efficient real-time detection of seizures would improve the ability of clinicians to quickly provide treatments and closely monitor patients during seizures, potentially improving patient outcomes through more timely interventions.

Note that although the non-limiting example embodiment of the study used iEEG, embodiments are not so limited. Some embodiments may use EEG data acquired using non-invasive scalp EEG techniques. Scalp EEG avoids the need for surgical implantation for monitoring and can be more easily performed in hospitals without access to neurosurgical centers. Scalp EEG can thus make embodiments accessible for a wider range of clinical and even home-based applications.

Certain examples can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), flash memory, and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by an electronic processor.

These computer readable program instructions may be provided to a processor of a general-purpose computer, special purpose computer, or other programmable data processing apparatus to produce a machine, such that the instructions, which execute via the electronic processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112 (f). However, for any claims containing elements designated in any other manner, it is intended that such elements are not to be interpreted under 35 U.S.C. § 112 (f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A computer-implemented method of automatically detecting a seizure of a patient, the method comprising:
obtaining patient electroencephalogram (EEG) data, wherein the patient EEG data represents an EEG of the patient for a plurality of channels, each channel representing a respective location in or on a brain of the patient;
evaluating, for each channel of the plurality of channels, a respective tendency to act as a sink, from which a plurality of tendencies to act as a sink are obtained, wherein each tendency to act as a sink of the plurality of tendencies to act as a sink quantifies a tendency of a respective location in or on the brain of the patient to act as a sink;
determining, for each tendency to act as a sink of the plurality of tendencies to act as a sink, a respective energy distribution according to frequency, from which a plurality of energy distributions according to frequency are obtained;
assessing an entropy of each energy distribution according to frequency of the plurality of energy distributions according to frequency, from which a plurality of entropy quantifications are obtained;
measuring, from at least one of the plurality of entropy quantifications, at least one sink tendency entropy drop value;
identifying, based on the at least one sink tendency entropy drop value, a presence of a patient seizure proximate to a time of a sink tendency entropy drop; and
outputting an indication of the patient seizure, wherein the indication of the patient seizure comprises an identification of the time.

2. The method of claim 1, further comprising treating the patient based on the indication of the patient seizure.

3. The method of claim 1, further comprising:
identifying, based on the plurality of entropy quantifications, at least one location in or on the brain of the patient that is epileptogenic of the patient seizure; and
outputting an identification of the location in or on the brain of the patient that is epileptogenic of the patient seizure.

4. The method of claim 3, further comprising treating the patient for epilepsy by one of surgical resection, laser ablation, or electrical stimulation of the location in or on the brain of the patient that is epileptogenic of the patient seizure.

5. The method of claim 1, further comprising:
reducing a dimensionality of the at least one sink tendency entropy drop value, from which a reduced dimensionality is obtained;
identifying a plurality of clusters of previous seizures of the patient in the reduced dimensionality;
classifying the patient seizure as being in one of the clusters of the plurality of clusters, from which a patient seizure classification is obtained; and
outputting an indication of the patient seizure classification.

6. The method of claim 1, wherein each of the plurality of tendencies to act as a sink comprises a respective dynamic network model sink index.

7. The method of claim 1, wherein the at least one sink tendency entropy drop value comprises a time under threshold value.

8. The method of claim 1, wherein the at least one sink tendency entropy drop value comprises an area under threshold value.

9. The method of claim 1, wherein the at least one sink tendency entropy drop value comprises an event activity metric.

10. The method of claim 1, wherein each of the plurality of energy distributions according to frequency comprises a spectral power density.

11. A system for automatically detecting a seizure of a patient, the system comprising: a non-transitory computer readable medium comprising instructions; and
at least one electronic processor that executes the instructions to perform operations comprising:
obtaining patient electroencephalogram (EEG) data, wherein the patient EEG data represents an EEG of the patient for a plurality of channels, each channel representing a respective location in or on a brain of the patient;
evaluating, for each channel of the plurality of channels, a respective tendency to act as a sink, from which a plurality of tendencies to act as a sink are obtained, wherein each tendency to act as a sink of the plurality of tendencies to act as a sink quantifies a tendency of a respective location in or on the brain of the patient to act as a sink;
determining, for each tendency to act as a sink of the plurality of tendencies to act as a sink, a respective energy distribution according to frequency, from which a plurality of energy distributions according to frequency are obtained;
assessing an entropy of each energy distribution according to frequency of the plurality of energy distributions according to frequency, from which a plurality of entropy quantifications are obtained;
measuring, from at least one of the plurality of entropy quantifications, at least one sink tendency entropy drop value;
identifying, based on the at least one sink tendency entropy drop value, a presence of a patient seizure proximate to a time of a sink tendency entropy drop; and
outputting an indication of the patient seizure, wherein the indication of the patient seizure comprises an identification of the time.

12. The system of claim 11, wherein the patient is treated based on the indication of the patient seizure.

13. The system of claim 11, wherein the operations further comprise:
identifying, based on the plurality of entropy quantifications, at least one location in or on the brain of the patient that is epileptogenic of the patient seizure; and
outputting an identification of the location in or on the brain of the patient that is epileptogenic of the patient seizure.

14. The system of claim 13, wherein the patient is treated for epilepsy by one of surgical resection, laser ablation, or electrical stimulation of the location in or on the brain of the patient that is epileptogenic of the patient seizure.

15. The system of claim 11, wherein the operations further comprise:
reducing a dimensionality of the at least one sink tendency entropy drop value, from which a reduced dimensionality is obtained;
identifying a plurality of clusters of previous seizures of the patient in the reduced dimensionality;
classifying the patient seizure as being in one of the clusters of the plurality of clusters, from which a patient seizure classification is obtained; and
outputting an indication of the patient seizure classification.

16. The system of claim 11, wherein each of the plurality of tendencies to act as a sink comprises a respective dynamic network model sink index.

17. The system of claim 11, wherein the at least one sink tendency entropy drop value comprises a time under threshold value.

18. The system of claim 11, wherein the at least one sink tendency entropy drop value comprises an area under threshold value.

19. The system of claim 11, wherein the at least one sink tendency entropy drop value comprises an event activity metric.

20. The system of claim 11, wherein each of the plurality of energy distributions according to frequency comprises a spectral power density.
